(19) United States
(12) Patent Application Publication
(10) Pub. No.: US 2009/0107498 A1
(43) Pub. Date: Apr. 30, 2009

(54) Respiratory Equipment and Method for Controlling Respiratory Equipment

(75) Inventors: Diana Plattner, Karlsruhe (DE); Bernd Scholler, Karlsruhe (DE); Matthias Schwaibold, Karlsruhe (DE)

Correspondence Address: FRIEDRICH KUEFFNER, 317 MADISON AVENUE, SUITE 910, NEW YORK, NY, 10017, US

(73) Assignee: WEINMANN GERÄTE FÜR MEDIZIN GMBH & CO. KG, Hamburg (DE)

(21) Appl. No.: 11/884,152

(22) PCT Filed: Feb. 10, 2006
(86) PCT No.: PCT/DE06/00253

§ 371 Date: Jul. 12, 2008

(30) Foreign Application Priority Data
Feb. 10, 2005 (DE) 10 2005 006 103.6

(51) Int. Cl. A61M 16/00

(52) U.S. Cl. 128/204.23

(57) ABSTRACT

The invention relates to a method and a device for operating or controlling respiratory equipment. According to the invention, a respiratory gas source, which can be connected to a patient interface, is coupled to a control unit. The control unit is configured to specify at least two different respiratory modes. The various respiratory modes are determined, selected and used for at least some respiratory disorders according to a current patient-specific requirement. The control unit is configured to automatically execute the control operations.

The invention concerns a ventilator that has a respiratory gas source that can be connected with a patient interface and a control unit designed for presetting at least two different ventilation modes.

The invention also concerns a device for controlling a ventilator, which has a respiratory gas source that can be connected with a patient interface and a control unit and in which at least two different modes of ventilation can be selectively activated by the control unit.

In obstructive sleep apnea syndrome, it is necessary to determine the individual pressure for CPAP therapy (CPAP=continuous positive airway pressure). The CPAP therapy is usually adjusted during a so-called titration night in the sleep laboratory. In this process, various methods are used to determine the optimum constant pressure for the patient for maintaining patency of the upper airways. The initiation of therapy is quite conservative at the present time. It is done almost exclusively in a clinical setting and not in a home setting. The optimum therapeutic CPAP can be determined by manual or automatic titration during polysomnography or respiratory polygraphy.

The usual titration method for finding the optimum CPAP pressure consists in incrementally increasing the CPAP pressure during nighttime polysomnography until episodes of obstructive apnea, hypopnea, and snoring no longer occur.

Alternatively, a CPAP device with automatic pressure regulation (auto-CPAP device) can be used in the hospital and/or on the patient at home over one or more nights. A pressure percentile can then be determined on the basis of the data recorded in the device and permanently set for long-term therapy, even though these devices are actually meant to supply the patient with the minimum ventilation pressure presently necessary.

At present, nasal CPAP therapy (nCPAP) with constant pressure with an individually adjustable rate of increase at the beginning of sleep is the method of choice in moderate to severe sleep apnea syndrome. If there is an especially high pressure requirement, this is poorly tolerated by the patient. The APAP or bilevel method is often used in such cases. In the bilevel method, a higher pressure is applied during inspiration, and a lower pressure is applied during expiration. This reduces the total pressure load. This makes it possible to adjust an additional fraction of the patients to a positive pressure method.

Many patients with obstructive sleep apnea have a strongly fluctuating pressure requirement, and “auto-CPAP devices” and “intelligent CPAP devices” can be of further help in these cases. The recognize at all times whether an obstruction is developing and promptly take action to control the situation, i.e., they increase the pressure to prevent closure of the airways.

Pressure oscillations integrated in the measuring process make it possible to differentiate so-called open and closed apneas.

The automatic CPAP pressure is varied as a function of indications of respiratory obstruction. The pressure is reduced to a minimum of 3 cm H₂O.

Another area of application of positive pressure ventilation is noninvasive ventilation, in which the ventilator supports the insufficient respiratory apparatus. Technical innovations, such as more convenient and manageable ventilators and especially leakage-reducing nasal masks, which replace tracheotomy in many cases, have resulted in improved therapeutic possibilities in recent years.

Indications for home ventilation include ventilation disturbances in neuromuscular diseases (e.g., central sleep apnea syndrome, primary alveolar hypoventilation syndrome, obesity-hypoventilation syndrome, CPAP-refractory obstructive sleep apnea syndrome, poliomyelitis and postpolio syndrome, spinal muscle atrophies, high cervical lesions, Guillain-Barré polyradiculitis, myopathies) and restrictive ventilation syndromes in diseases of the skeleton, pleura, and lungs (e.g., kyphoscoliosis, status after extensive lung resection, extensive scarring changes of the pleura and lung, status after thoracoplasty).

In the titration of the optimum therapy pressure for the treatment of obstructive sleep apnea syndrome, signal magnitudes are usually sought which allow early detection of an obstruction. For example, the development of a plateau of the inspiratory flow signal is seen as a sensitive sign of a mild obstruction. Raising the CPAP pressure counteracts the increased resistance of the upper airways and leads to normalization of the flow contour.

A reliable but highly invasive method for determining the level of obstruction is the combined measurement of the esophageal pressure and the respiratory flow.

A comfortable alternative is measurement of the respiratory impedance.

Automatic CPAP titrations are based on respiratory characteristics which characterize the severity of the upper airway obstruction.

The incremental adjustment of the CPAP pressure or of the ventilation parameters during a nighttime polysomnography (PSG) for optimum adjustment of the patient is not free of subjective criteria. For the standardization, reproduction, and reconstruction of the automated procedure, it would be a great advantage if a sufficiently good ventilation adjustment were possible by means of established algorithms.

Another advantage can consist in applying the pressure that has been adjusted by computer assistance only during the obstruction or reduced ventilation and significantly lowering the nCPAP pressure, e.g., when the patient is awake or during phases of unobstructed breathing. This could also make it possible to improve acceptance of the method and possibly also the sleep quality even during the titration.

Various measured quantities, such as oxygen saturation and respiratory flow, can be considered for the automatic titration. For example, an increase in resistance of the upper airways can lead to a flattening of the contour of the inspiratory flow/time curve. Increased resistance in the region of the extrathoracic airways can thus already be recognized before the occurrence of relevant hypopneas from the pattern of the inspiratory flow or other physiological or instrument signals.

The objective of the present invention is to design a device of the aforementioned type in a way that assists automatic titration.

In accordance with the invention, this objective is achieved by designing the control unit for the automatic determination, selection, and application of the various ventilation modes in at least some respiratory disorders according to a current patient-specific requirement.

A further objective of the present invention is refinement of a method of the aforementioned type in such a way that the performance of an automatic titration is assisted.

In accordance with the invention, this objective is achieved by automatically determining, selecting, and applying the various ventilation modes in at least some respiratory disorders according to a current patient-specific requirement.

The adjustment can thus also be carried out independently of the premises, e.g., with a patient at home. In addition, the device can be used via a data processing program for remote adjustment, therapy supervision, and autotitration, such that the program carries out an automatic detection of the connected device during the establishment of communication, and all titration parameters can be adjusted.

This makes it possible to save a good deal of time and expense, since more patients can be simultaneously monitored by fewer personnel. Moreover, better at-home adjustment results can be obtained due to the patient's being in familiar surroundings.

Furthermore, a titration/therapeutic device of this type makes it possible to close the gap that presently exists between diagnosis and therapy.

Specific embodiments of the invention are schematically illustrated in the drawings.

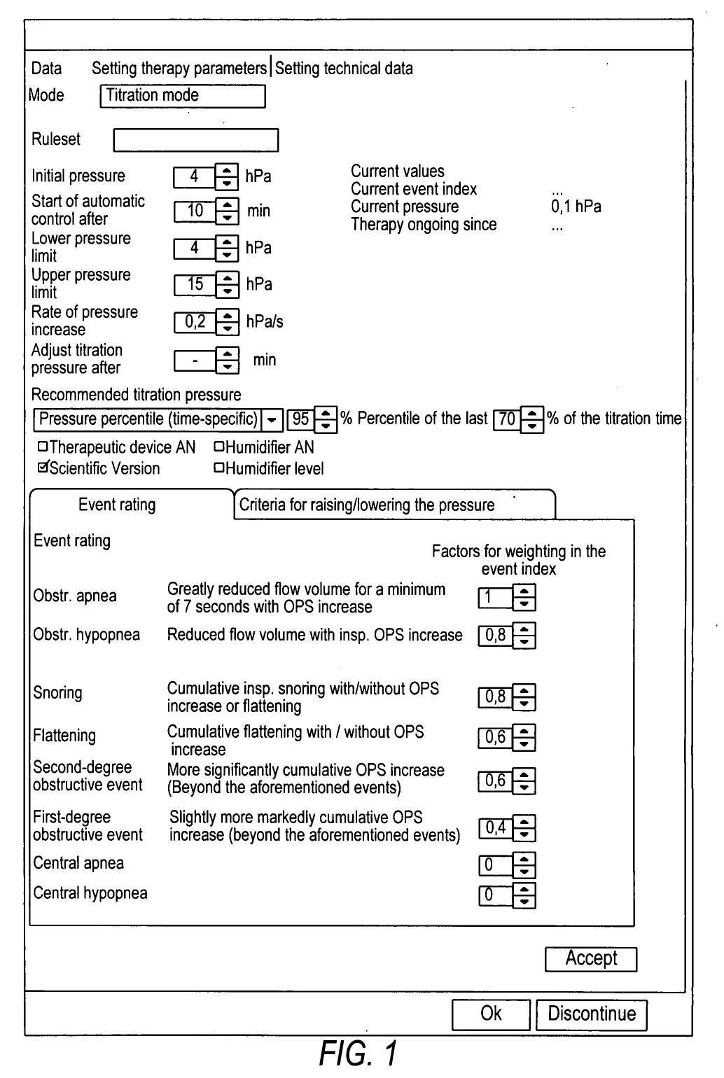

FIG. 1 shows an operating screen for inputting and displaying device parameters.

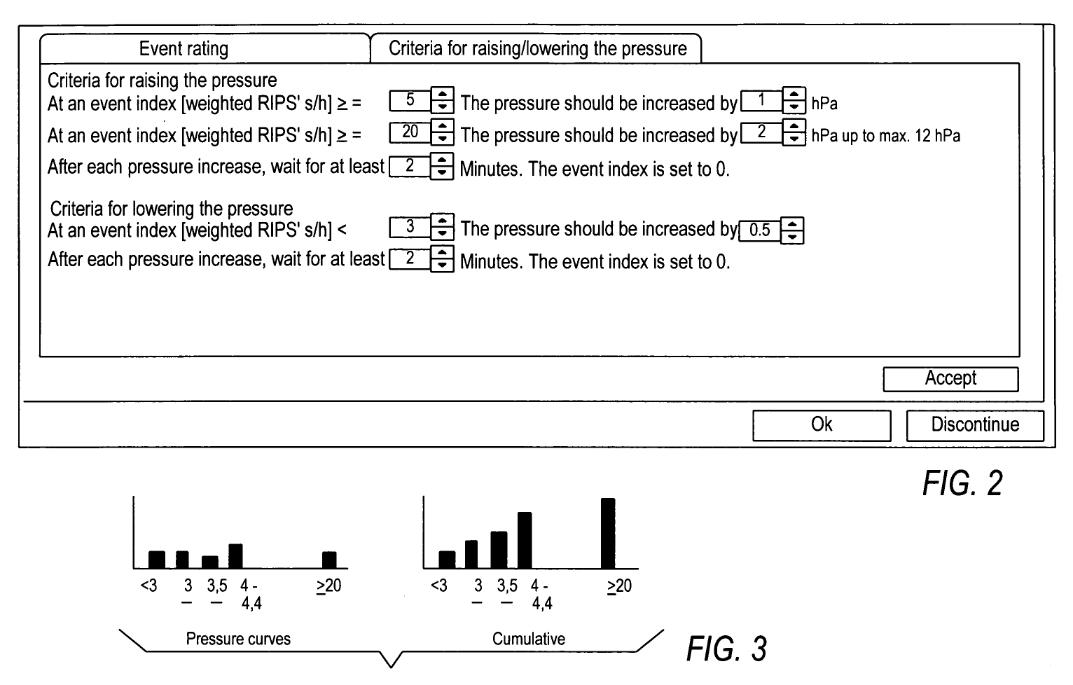

FIG. 2 shows another operating screen for evaluating events.

FIG. 3 shows cumulative pressure curves.

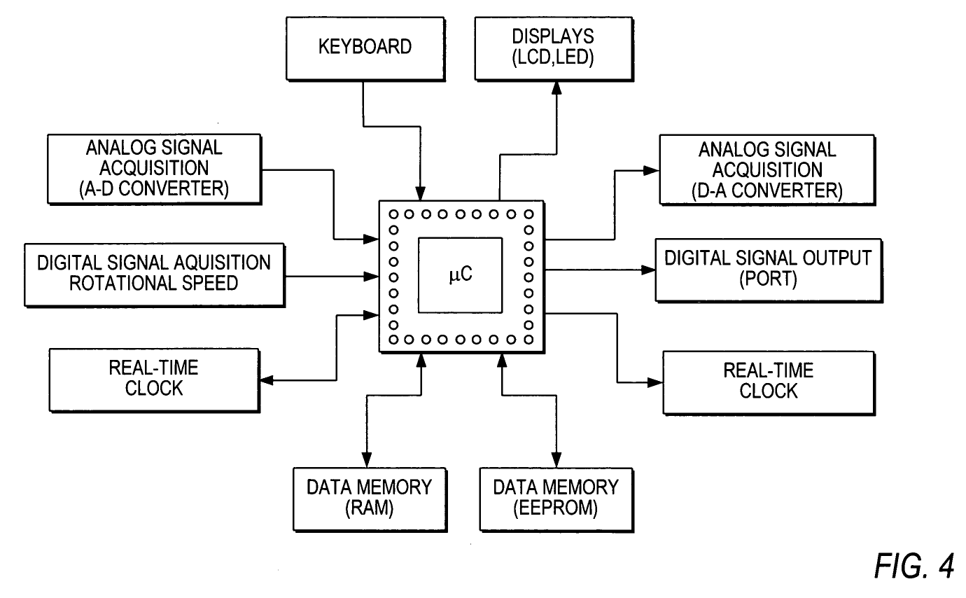

FIG. 4 shows a hardware structural diagram.

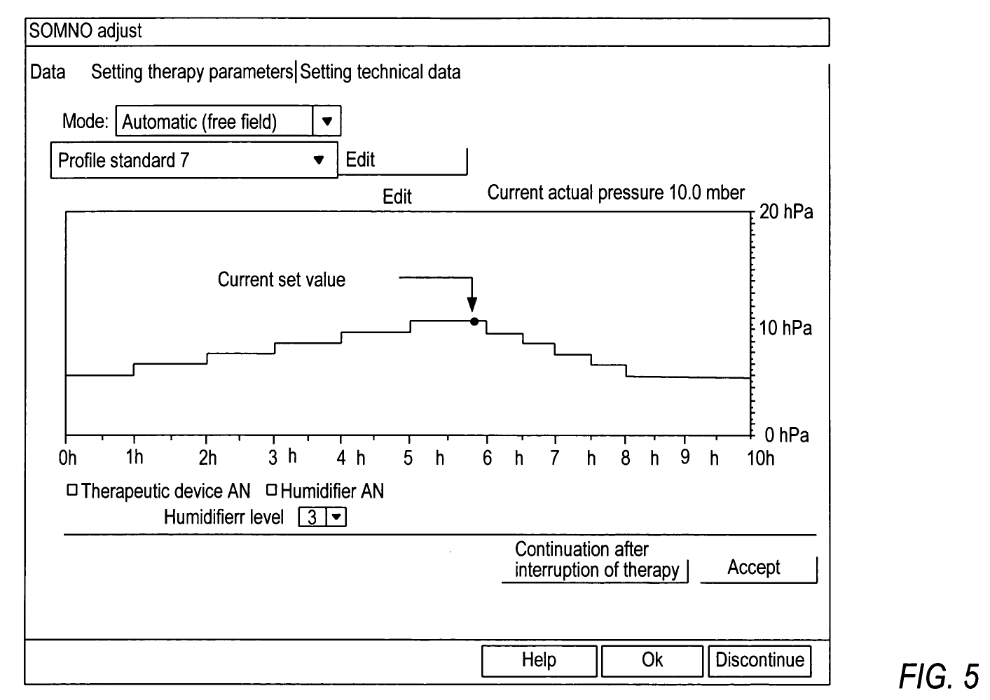

FIG. 5 shows a displayed pressure curve.

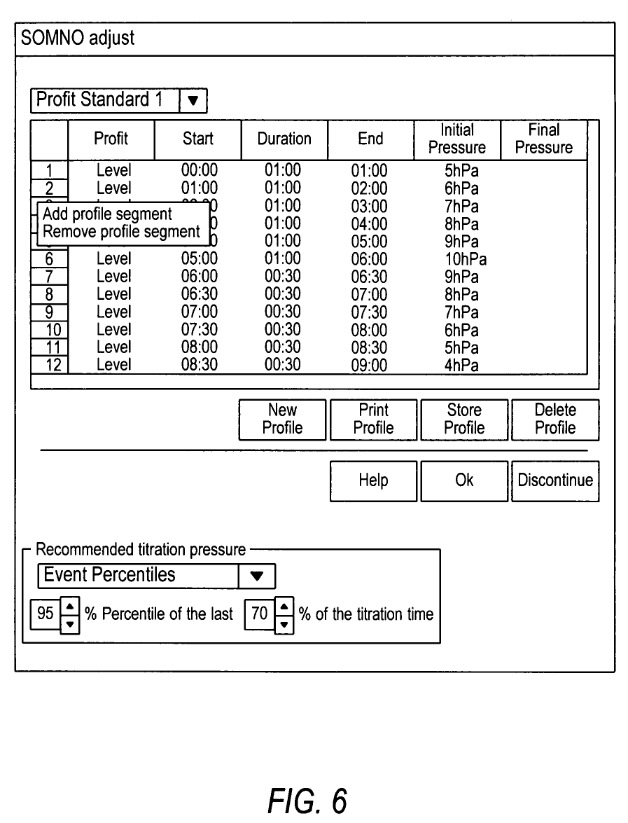

FIG. 6 shows a screen display of a summary of results.

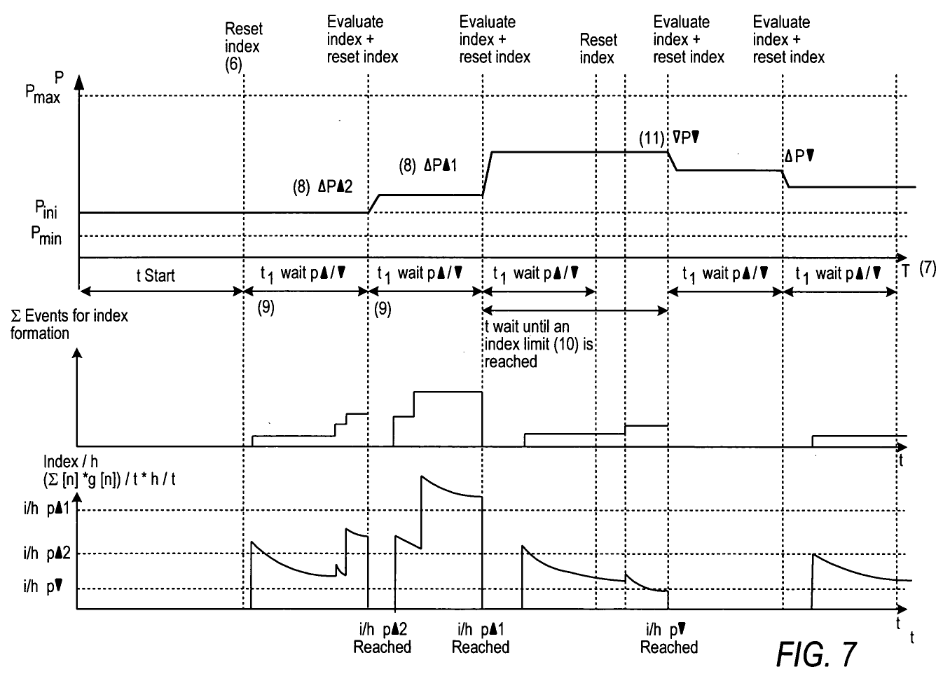

FIG. 7 shows signal curves for carrying out the titration.

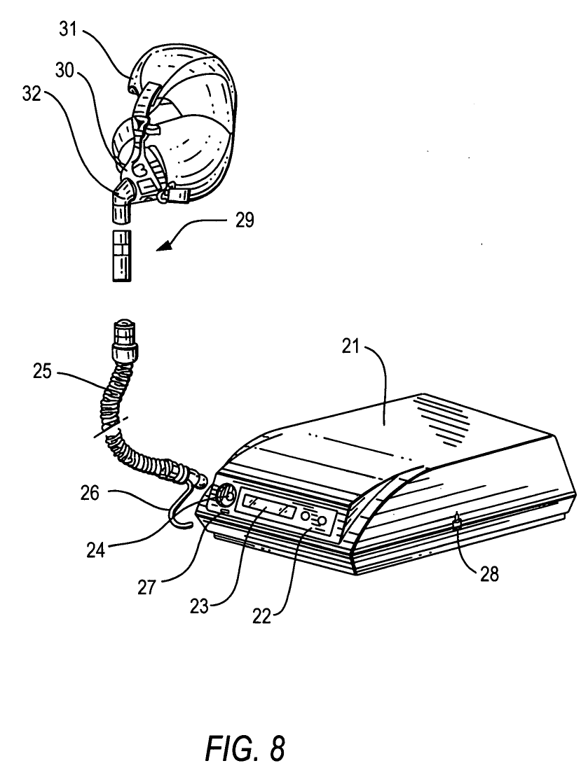

FIG. 8 shows a perspective view of a ventilator with respiratory hose and respiratory mask.

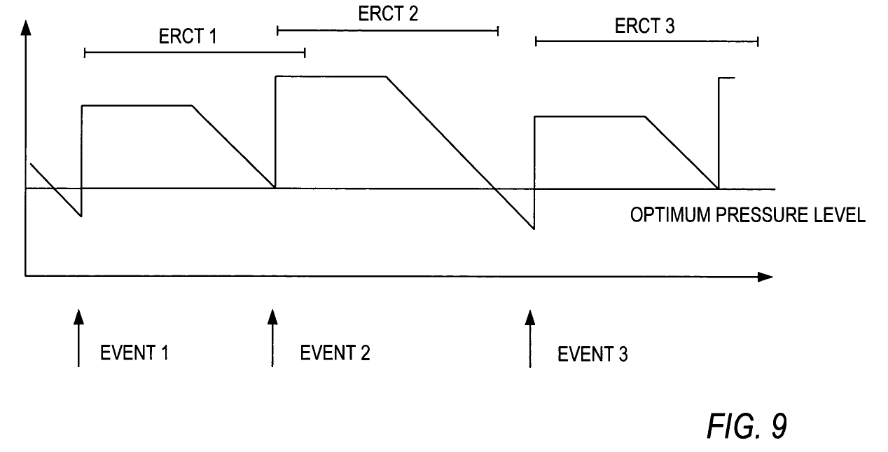

FIG. 9 shows a pressure curve to illustrate a control characteristic of the ventilator.

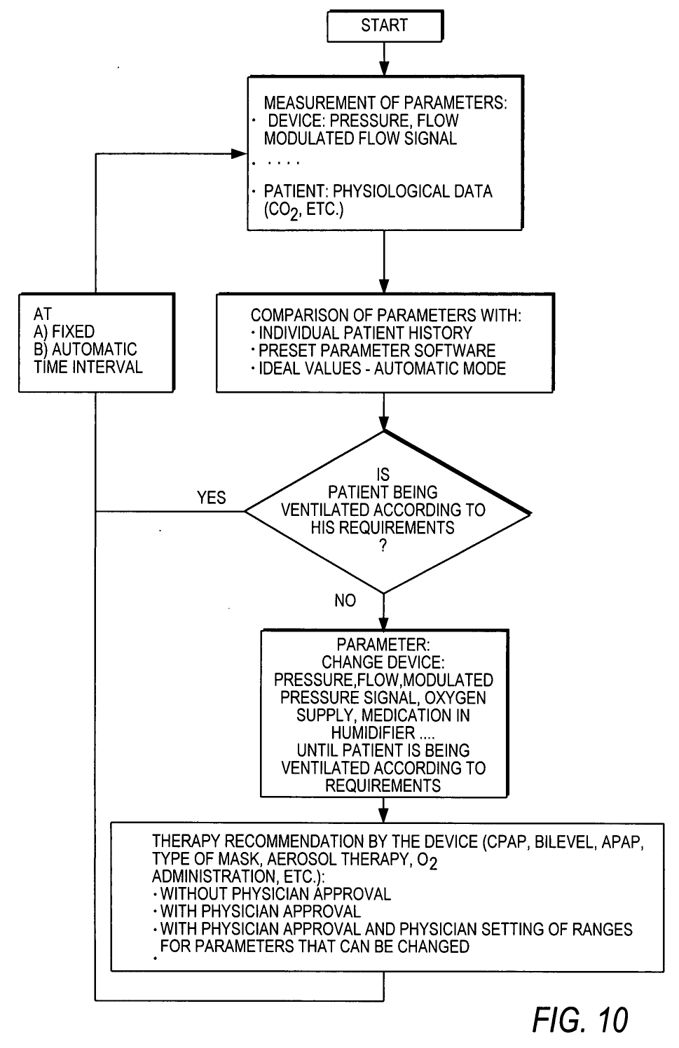

FIG. 10 shows a flowchart for carrying out the device control.

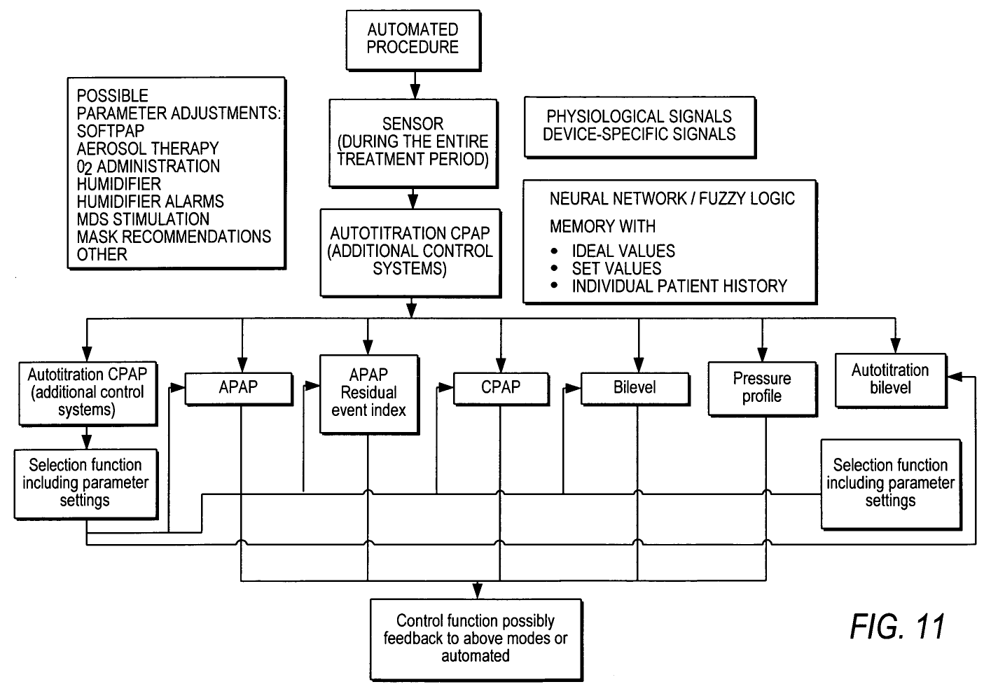

FIG. 11 shows a block diagram for the structured representation of the device control system.

The method provides an analysis by at least one sensor, which detects physiological and device-specific parameters, such as compliance, resistance, blood values (O₂, CO₂, HB, CO, etc.), pressure, flow, MDS, temperature, motor output, fan flow, and fan voltage, and then generates signals and transmits them to an analyzer. The analyzer uses a selection function to compare the signal information thus received with information (erasable, modifiable) stored in a memory device. This information is based on an individual patient history and/or is derived from evaluated patient histories, which are linked with one another and evaluated by means of fuzzy logic and/or neural networks. The analyzer makes a mode assignment, and on the basis of this mode assignment, including parameter adjustment, a control unit is activated in such a way that effectors (displays, fans, etc.) are triggered.

Alternatively (specifically for APAP titration and bilevel titration), the analysis can also be performed by a person, who independently makes the mode assignment.

In an especially preferred form, the design provides that, in the diagnostic phase, the physician can select and adjust an existing ventilation form/type (automatically with permanently set pressure profiles, CPAP, APAP, bilevel, autotitration CPAP, autotitration bilevel, etc.). However, an automatic option can also be selected, in which the device starts the ventilation independently on the basis of various parameters supplied and measured by the patient.

In the appropriate modes, after the titration phase, the device outputs a therapy recommendation, which either is carried immediately, i.e., without further consultation with the physician, or must be confirmed by the physician/user. Furthermore, other recommendations are issued regarding possibly necessary oxygen or aerosol therapy and/or a mask or human/machine interface to be used.

In another especially preferred embodiment, after the device has been started, current device parameters, such as pressure, flow, MPS, and temperature, and current patient parameters, such as compliance, resistance, blood values (O₂, CO₂, HB, CO, etc.), are measured. This is illustrated in FIG. 10. In an additional step, these current parameters are compared with the data of an individual patient history, values preset in the parameter software and/or in an automatic mode with ideal values, and a rating of the ventilation required for the patient is calculated. This is shown in FIG. 11.

The device generates a recommendation for ventilation according to the patient's requirements. This recommendation to the physician pertains to the form of ventilation, such as CPAP, APAP, Bilevel, together with the associated ventilation parameters, and the type of additional, supporting functions of the device, such as O₂ administration, monitoring and storage of patient data, aerosol therapy, humidifier, full-face mask, etc. The physician can then accept the therapy recommended by the device, or he can adapt and adjust the ventilation parameters in ranges in which the parameters can be independently varied by the device.

In his initial setting of the device, the physician can also provide the device with almost complete autonomy for changing the parameters, so that after the therapy recommendation has been made, the device automatically jumps to the recommended mode and provides the patient with further ventilation in this mode.

The device continuously monitors and modifies the parameters of the device until the patient is being ventilated according to his specific requirements. In this connection, it is possible to work with either a fixed or an automatic interval or to generate the output of a new recommendation according to the severity of the respiratory disorder.

When ventilation is being carried out in conformity with the patient's specific requirements, the cycle can be started anew according to a fixed or an automatic cycle.

The device and the method utilize a technology with continuous automatic event detection derived from MPS-specific (modulated pressure signal), flow-specific and pressure-specific quantities, and with artifact detection relevant to ventilation pressure in order to control the applied ventilation pressure—but in this case not only to control a ventilation pressure but also to determine a wide variety of parameters that are important to the ventilation. The events and artifacts arising during respiration are supplied to a ruleset that forms an event index in the titration modes during the application of ventilation in order to determine the therapeutic pressures, among other ventilation parameters, that are optimum and sufficient for the patient.

The APAP devices are equipped with compressed gas sources, which are connected with a patient's airways by a ventilation hose and a ventilation mask. The compressed gas source is usually controlled in such a way that a specific pressure control is carried out as a function of a measured respiratory parameter, for example, the flow or the pressure.

In another proposed embodiment, especially for ambulant titration and/or titration for a user at home, a diagnostic device records physiological signals of a patient and supplies them to an evaluation unit. The evaluation is performed automatically according to definable criteria, such as AHI.

The diagnostic device signals a recommendation on the basis of the result of the evaluation, for example, it asks the patient to use a ventilator, e.g., a CPAP, APAP, or bilevel device.

The diagnostic device communicates with the ventilator in the form of a titration. Each device can influence the other. It is proposed that preferably the diagnostic device should control the ventilator.

A setting of the ventilator that is optimal for the patient is determined and/or maintained by the adjustment of the ventilation parameters.

In another embodiment of the invention, if the need for therapy is determined, the diagnostic device triggers an alarm to waken the patient, who can then start the ventilator.

In the second part of the night, the diagnostic device continues to record signals and controls the ventilator in the form of a titration in such a way that optimum therapy for the patient is determined and/or maintained.

This makes it possible to establish a diagnosis and to adjust the therapy in the patient's home in only one night.

In a supplementary embodiment, the physician receives a diagnosis and therapy report from the same night.

Another great advantage of the present invention is the optional coupling of the therapeutic device with the diagnostic device or with PC software of the diagnostic device for configuration of the therapeutic device or feeding signals or detected events or internal states of the therapeutic device into the diagnostic device. In this regard, feeding can be accomplished online or offline at a very fast data transmission rate, so that the therapeutic device, coupled with a device for detecting polysomnographic data, can perform diagnostic procedures with respect to sleep stages and position, hypoxia, cardiac disturbances, hypercapnia/hypocapnia, leg movements, respiratory exertion, mouth and mask leakage, mouth exhalation, signs of obstruction, such as snoring, flow limitations, obstructive apneas and hypopneas, hyperventilation and hypoventilation, central respiratory disturbances, etc.

The device operates both on a stand-alone basis and in conjunction with computer programs for remote adjustment and data visualization and data processing. Depending on the mode of the device which has been set, the event-dependent pressure adjustment is carried out. However, the event detection and storage is identical in all modes of the device.

The device has the following modes:
Mode CPAP autotitration (various rulesets can be selected)
Mode pressure profile (fixed and adjustable pressure profiles)
Mode CPAP (for the monitoring night or manual titration)
Mode APAP
Mode bilevel (optional)
Mode APAP residual index
Mode bilevel autotitration (optional)
Mode automatic (device selects the ventilation mode independently or as a result of the data delivered by the diagnostic device or by the sensor and analyzer)(optional)

Mode Selection with Effector Activation

In the CPAP autotitration mode, the pressure adjustment is carried out on the basis of events in accordance with the selected ruleset. The ruleset defines the weighting of the events that have occurred (for the calculation of the event index) and the parameters of the pressure adjustment.

A fixed ruleset (AT1) and a user-defined ruleset (AT2) are provided as a preferred embodiment. The user can activate the fixed or user-defined ruleset in standby (offline) operation. The control system parameters can be displayed and modified (only the user-defined ruleset) in the standby (offline) operation with PC software (via the serial interface).

In this regard, in an especially preferred embodiment, in the ruleset, which can also be adjusted via the interface, the user can preset or adjust for the ventilation, among other things, the period or various periods for the start of titration, the end of titration, minimum delay times for pressure adjustment and the following parameters: initial pressure, start of automatic control, upper and lower pressure limit, rate of pressure increase, rule for the recommended titration pressure, and split-night option.

In accordance with FIG. 1, the initial pressure can be adjusted between 3 and 80 hPa in increments of 0.1 hPa, the start of automatic control between 0 and 500 minutes in intervals of 5 minutes, the lower pressure limit between 3 and 80 hPa in increments of 0.1 hPa, the upper pressure limit between 3 and 80 hPa in increments of 0.1 hPa, and the rate of pressure increase between 0.1 and 0.6 hPa/s in increments of 0.1 hPa/s. In this regard, the current event index [/h] and the current pressure can be displayed by the software.

In addition, in an especially preferred embodiment, the weighting factors and control criteria for raising and lowering the pressure are also part of the ruleset. While all rulesets can be stored in the software, only a user-defined ruleset and a default ruleset are stored in the therapeutic device. The default ruleset in the therapeutic device is not overwritable. Changes in the settings in the ruleset in the therapeutic device can be made only on the user-defined ruleset.

In an especially preferred form of the autotitration mode, a split-night option is implemented, i.e., the titration is interrupted after a defined time, the recommended titration pressure is calculated, and then the therapy is carried out with the determined pressure. In this process, it is necessary not to fall below a minimum adjustment time, and the adjustment range can be increased in increments (e.g., adjustment range of 60-600 minutes and no automatic termination of the titration).

In a preferred form, the resulting CPAP is stored in the device calendar and can be retrieved by the physician. The device can be operated, for example, by operator interfaces, which can consist of keys, turnable control knobs, touchscreens, and/or other operating elements. The preset pressure variations and rulesets can only be activated on the device and not edited. To edit the titration parameters, the pressure variation, or to allow manual pressure adjustment, a connection with a PC and with software is necessary via an interface.

In an especially preferred form, the recommended titration pressure can be calculated as a time-related pressure percentile or an event-related pressure percentile. In this regard, a value can be preset for the percentile and a percentage value can be preset in well-defined intervals, starting at a base value (e.g., 10%) of the titration time. It is also possible to adjust or determine the pressure in permanently preset adjustment increments or, optionally, in variable adjustment increments (e.g., 5%) by the output of the final pressure or median of the last percentage of the titration time.

The initial pressure, the start of automatic control after “X” minutes, the lower pressure limit, the upper pressure limit, the rate of pressure increase, the titration pressure adjusted after “X” minutes, the duration of therapy, and the recommended titration pressure (rule and value) can be recorded for a printable “titration report” and on the screen display. In the adjustments of the pressure limits, an intelligent modification is used: If the initial pressure increases above the lower and/or upper pressure limit, there is an automatic increase in the lower and/or upper pressure limit to the initial pressure. If the initial pressure decreases below the lower pressure limit, there is an automatic decrease in the lower pressure limit to the initial pressure. If the lower pressure limit increases above the initial pressure and/or the upper pressure limit, there is an automatic increase in the initial pressure and/or the upper pressure limit to the lower pressure limit. If the upper pressure limit decreases below the initial pressure and/or the lower pressure limit, there is an automatic decrease in the initial pressure and/or the lower pressure limit to the upper pressure limit.

In the adjustments of the time for the start of automatic control and the split-night time, an intelligent modification is also used: If the start of automatic control rises above the time for the start of the adjustment of the titration pressure, the time for the adjustment of the titration pressure is automatically adapted.

If the time for the adjustment of the titration pressure falls below the time for the start of automatic control, the time for the start of automatic control is automatically lowered to the selected amount.

If the nasal mask is removed during the autotitration (e.g., the patient goes to the bathroom), then the event evaluation, index formation, and pressure adjustment are halted, but the data storage continues. A notice appears on the display (e.g., “PAUSE”). The fan is deactivated (or operated at minimal speed). If the interruption lasts for less than an adjustable or fixed time limit, the autotitration (event detection and pressure adjustment) is then continued. If the interruption lasts longer, the device is shut down.

The index formation is reinitialized after an interruption that is less than an adjustable or fixed time limit.

In the pressure profile mode, the pressure adjustment can be carried out according to the selected pressure profile. Events do not result in a pressure adjustment. Fixed and user-defined profiles are stored. These stored profiles are shown, for example, in FIG. 5 and FIG. 6.

The user can activate one of the two fixed pressure profiles or the user-defined profile in standby (offline) operation and in online operation. The pressure profiles can be displayed and modified (only user-defined pressure profiles) in standby (offline) operation and in online operation with PC software via the serial interface. If the profile duration is shorter than the therapy duration, the therapy is continued with the last profile pressure.

In this mode, the titration pressure calculation can also be selected. Thus are in therapy checks using profiles adjusted to the patient around the previous therapy pressure value for the exact evaluation of pressure sufficiency possible.

In one embodiment of the invention, to realize better visualization and reconstruction of the currently prevailing pressure, a tracking cursor (e.g., a dot, crosshair cursor, etc.) is seen on the display screen of the PC software and/or on the display of the operating element, which shows the current set pressure and position in the sequence on the pressure variation during the night.

In the APAP mode, there is an immediate pressure adjustment on the basis of therapy-relevant individual events. With the mask open and the automatic system activated, the device shuts off after 5 seconds.

APAP Residual Event Index

Until now, the procedure has always been to raise the pressure with every obstructive event, hold it constant for a while and then slowly lower it again; this principle is used in virtually all APAP devices.

The amount of time that elapses before the initial pressure (the pressure at the beginning of the event) is reached again is called the “event reaction cycle time” (ERCT). The event has influence on the pressure during this period of time.

As FIG. 9 shows, if the next event follows faster than the ERCT, the device responds again but this time at an even higher initial pressure. If the next event again follows faster than the ERCT of the second event, the pressure is raised still higher. A net pressure increase thus occurs.

If the next event follows more slowly than the ERCT, the pressure has already fallen below the initial pressure. Although an increase then occurs in response to the event, the increase is to a lower level than the increase in response to event 1. If the next event again does not occur until later than the ERCT, the device is at an even lower pressure level. There is a net pressure drop.

If the next event follows exactly at the completion of the ERCT, the device is again exactly at the initial pressure. It thus swings back and forth between two pressure levels, and the pressure remains net constant.

Skillful selection of the ERCT thus makes it possible to “tune in” a stable operating point, i.e., to adjust a time between two events at which the system remains stable. The reciprocal of the ERCT is the event index obtained in this stable point. If a long-term stable residual AHI of 5 is desired under the therapy, it is thus necessary to adjust the ERCT to 12 minutes.

Naturally, since the patient is not a totally stable system that is invariable with respect to time, corresponding automatic pressure control does not yield an AHI of exactly 5; however, it is a rather robust starting point for the adjustment of the lowering delay times and lowering rate of an APAP device.

Four parameters can thus be adjusted: lowering delay times and rate, the pressure increase when an event occurs, and the ERCT.

Most APAP devices drop much too soon and therefore depend on the fact that the patient develops several mild obstructions, to which the device can already respond again, before he develops another severe, AHI-relevant obstruction.

However, since there is no knowledge at all about whether the patient will at present develop 4 mild obstructions per severe obstruction or 20 or none at all, a highly variable AHI is obtained with most APAP devices.

The above approach, on the other hand, “guarantees” (as long as the patient is stable) an AHI≦5, even with no mild obstructions at all.

In the CPAP mode, pressure adjustment is not carried out. The device makes the adjusted pressure constantly available. With the mask open and the automatic system activated, the device shuts off after 5 seconds.

In an especially preferred form, a selectable, so-called softstart mode is implemented, which, when the device is activated, automatically slowly raises the pressures (chiefly the CPAP pressure) within a specific, adjustable time period to the desired level (CPAP mode) or the desired pressure range (APAP mode). This option can be deactivated in the autotitration mode.

In the bilevel mode, pressure adjustment is not carried out. The device makes the adjusted inspiratory and expiratory pressure constantly available, together with the associated ventilation parameters (e.g., rate of pressure change, assisted, controlled, assisted/controlled ventilation forms).

In the bilevel titration mode, adjustments of pressure and other parameters are made as a function of a large number of respiratory events that have occurred. In this regard, volume-controlled or pressure-controlled ventilation can be carried out. With the mask open and the automatic system activated, the device shuts off after 5 seconds.

In the titration modes, the aforementioned weighting of events that have occurred during respiration and of artifacts relevant to ventilation pressure, which are relevant for the airway disturbance and its therapy, and artifact sensibilities of the user can be preset or adjusted with quantities for the ventilation.

The following are examples of artifacts of this type which are relevant to ventilation pressure: parameters derived from MPS-related, flow-related, and pressure-related quantities, such as change in patient position, mouth and mask leakages, mouth expiration, speaking, coughing, clearing the throat, and swallowing.

To detect events and artifacts, the following signals, among others, are used:

In another especially preferred embodiment, the (device) flow signal is determined from the (fan) speed and the current (mask) pressure. This calculation is carried out at 100 Hz (10 ms).

The MPS is produced by bandpass filtering (IIR filter) of the MPS raw signal. In another especially preferred embodiment, this filtering allows transmission of frequencies of 19-21 Hz and attenuates all other frequencies.

The filtering is carried out at 500 Hz (2 ms). The signal is then rectified, and the maximum of the oscillation is determined over a period of 50 ms. After 50 ms (25 values), the maximum is stored as the new MPS value, and the maximum is reinitialized.

The snoring signal is produced by bandpass filtering (IIR filter) of the MPS raw signal. In another especially preferred embodiment, this filtering allows transmission of frequencies of 65-190 Hz and attenuates all other frequencies.

The filtering is carried out at 500 Hz (2 ms). The signal is then rectified.

The present invention is intended to guarantee optimum device control even if disturbances occur.

In accordance with the invention, this goal is achieved by virtue of the fact that the control system has an analyzer for detecting at least one artifact and by virtue of the fact that the analyzer is coupled with the control system in such a way that, when the artifact is detected, an erroneous pressure change preset by the adaptation device is avoided.

The device of the invention and the method of the invention utilize the fact that specific artifacts lead to typical effects on the measured respiratory parameter. The corresponding typical variations of the respiratory parameter with respect to time that are assigned to a specific artifact allow an automatic evaluation of the signal pattern with respect to the given variations and thus an identification of the given artifact by control technology.

Corresponding specific artifacts that can be recognized by the signal pattern are, for example, mouth expiration, mouth breathing, leakage, swallowing, speaking, or coughing. With automatic detection of such events, it is possible to modify the pressure control in such a way that those measurement parameters that are evaluated in a normal state of the control system for a pressure increase or a pressure decrease and that can no longer be reliably evaluated when the given artifact occurs are not taken into consideration for the duration of the occurrence of the artifact.

A basic process sequence in the detection and consideration of artifacts by control technology can be carried out in such a way that the detection of inspiratory and expiratory phases is carried out first by evaluating a signal related to the flow or by evaluating a signal related to pressure fluctuations. A measurement is made of the amplitude or power of expiratory pressure fluctuations, which are either produced by the device or arise within the patient. The pressure fluctuations produced by the device can be formed by a modulated pressure signal, for example an MPS signal or an FOT signal. Pressure fluctuations produced by the patient can be, for example, hissing noises during mouth expiration or leakages.

After the measurement has been made, the measured amplitude or the measured power is compared with a reference value derived from preceding expiratory phases or inspiratory phases. If a significant change is determined, one or more types of artifacts are recognized.

Optionally, it is possible to distinguish the artifacts by the magnitude of the change, by the number of consecutive breaths with the change, or by an evaluation as to whether the given change occurs only in the expiratory phases or also in the inspiratory phases compared to the preceding breath.

It is also optionally possible to achieve a further increase of the analytical quality by evaluating the flow variation if, for example, the expiratory half wave is absent during mouth expiration.

The amplitude of the pressure oscillation measured during expiration is compared with the amplitude of the pressure oscillation measured during inspiration. In this connection, the mean values of the two respiratory phases are compared.

If the expiratory value is greater than the inspiratory value by at least a preset, first amount, then an individual expiratory artifact, for example, swallowing, coughing, or speaking, is detected.

If the expiratory values are greater than the inspiratory values by at least a preset, second amount for a preset number of breaths, then mouth breathing or mouth expiration is detected.

Depending on the result of the evaluation, the evaluation of the inspiratory flow volume is suppressed, and thus the detection of hypopnea is suppressed, and likewise the evaluation of the inspiratory amplitude of the pressure oscillation in conjunction with the oscilloresistometry is suppressed, since during mouth breathing or when an expiratory artifact occurs, a corresponding response is unreliable.

In accordance with another embodiment, the natural pressure oscillations are measured in a specific frequency band by bandpass filtering of the measured pressure signals in order to detect snoring or hissing noises. If there is an expiratory increase, mouth expirations are detected, and if there is an inspiratory or expiratory increase, leakage is detected. In each case, there is also suppression of events or shifting of threshold values, for example, for the detection of snoring.

In a typical process sequence, the control system is designed for carrying out APAP ventilation.

In accordance with one embodiment, it is provided that the analyzer is designed for the evaluation of a flow variation.

Furthermore, it is also contemplated that the analyzer be designed for the evaluation of a pressure variation.

In one variant of the method, the analyzer is designed for the evaluation of inspiratory phases.

It is also possible for the analyzer to be designed for the evaluation of expiratory phases.

In accordance with a simple evaluation principle, the analyzer is designed for the evaluation of amplitude values.

It is also possible for the analyzer to be designed for the evaluation of power values.

In accordance with another embodiment, it is provided that the analyzer has a reference value comparator.

Frequency-dependent signal analysis is assisted by carrying out bandpass filtering of the measured pressure signal.

In particular, it is proposed that a frequency band in the bandpass filtering be defined in such a way that an amplitude of a volume oscillation produced by the device is measured.

An excitation signal adapted to the bandpass filtering can be supplied by generating the volume oscillation by a diaphragm pump.

In accordance with another embodiment, it is possible to generate the volume oscillation by a fan drive.

A good compromise between simple hardware realizability and good analyzability of the excitation signal consists in the generation of a volume oscillation with a frequency of about 20 Hz.

In accordance with a typical evaluation process, it is provided that an evaluation is carried out in such a way that an expiratory constriction of the airways is detected by an expiratory increase in the pressure oscillation amplitude relative to a reference value.

A special variant of the detection of a problem consists in carrying out an evaluation in such a way that individual or cumulative expiratory constrictions are rated as artifacts.

In an evaluation of the frequency of occurrence of artifacts, it is possible to evaluate the quality of the type of mask that is being used. This allows quality control in both APAP applications and CPAP and bilevel applications.

The events already mentioned above can be: parameters derived from MPS-related, flow-related, and pressure-related quantities, such as obstructive/central apneas, obstructive/central hypopneas, snoring with and without obstruction of the airways, flattening of the respiratory flow, mild and severe obstructions, and respiratory resistance and compliance.

The recording of events for therapy monitoring is, for example, 42 days.

In another especially preferred embodiment, all events or only individually integrated events are detected as follows:

Obstructive Apnea:

Greatly reduced flow volume for at least 7 seconds (and 2 breaths) with an MPS increase. (MPS limiting value rises with pressure; starting at 12.5 hPa (value adjustable), all apneas are rated as central; starting at 7 hPa (value adjustable), the occurrence of the MPS increase must not be delayed until the end of the apnea.)

Obstructive Hypopnea:

Reduced flow volume (2 consecutive inspirations, max. 60% (value adjustable) with OE1 or OE2 (in intervals with many central events: only with OE2).

Obstructive Snoring:

Cumulative snoring (3 (number adjustable) consecutive inspirations) with OE1, OE2, or with flattening. The snoring signal exceeds the defined threshold value for a well-defined minimum duration (0.3 s (value adjustable)) during the inspiration. Snoring is stored in the titration data (as an event) and in the weekly compliance (as variation, epoch with/without snoring).

Obstructive Event of Second Degree:

Significant cumulative (3 (number adjustable) breaths) inspiratory MPS increase.

Obstructive Flattening:

Cumulative and strongly pronounced flattening (at least 3 of 6 inspirations (number adjustable)), at least one inspiratory MPS increase.

Primary Snoring:

Cumulative snoring (3 (number adjustable) consecutive inspirations) without inspiratory MPS increase. The snoring signal exceeds the defined threshold value for a well-defined minimum duration (0.3 s (number adjustable)) during the inspiration. Snoring is stored in the titration data (as an event) and in the weekly compliance (as variation, epoch with/without snoring).

Flattening:

Cumulative flattening (3 of 6 (number adjustable) inspirations).

Obstructive Event of the First Degree:

Slightly pronounced cumulative (2 breaths (number adjustable)) inspiratory MPS increase.

Central Apnea:

Greatly reduced flow volume for at least 10 seconds without MPS increase.

Central Hypopnea:

Reduced flow volume (at least 2 consecutive inspirations and at least 10 s (number and minimum duration adjustable)) without MPS increase.

Artifact:

Time criteria and unrest criteria, e.g., with respect to the flow signal and/or the MPS. Events are no longer detected. The index formation continues unchanged.

Mask Open:

Set pressure cannot be reached. Events are no longer detected. Set pressure and index are frozen.

Mouth and Mask Leakage:

Loss flow above 0.33 L/s (=20 L/minute). Events are no longer detected. The pressure is held constant or lowered in accordance with the index criteria, as long as it is above 10 hPa (value adjustable).

Mouth Expiration:

Strong expiratory MPS increases (minimum 3×(number adjustable)). Obstructive events and episodes of hypopnea are no longer detected. The index formation is frozen.

MPS Artifact:

Expiratory MPS increase. Obstructive events and episodes of hypopnea are no longer detected. The index formation is continued unchanged.

Other applications are respiratory events that are relevant to ventilation parameters, such as arousals and hyperventilation (inspiratory volume increased).

For better detection and adjustment of the devices individually to the patient, the user can additionally define the following in the ruleset of the titration modes:

Event index limiting values, which consist of the aforementioned respiratory events and their rating and lead to a pressure adaptation when the values rise above or fall below these values within the period of time established above, absolute pressure increases of the device or pressure drops when the previously established event index limiting values are reached.

The following weighting factors are defined for the rating of the events:

Weighting Factor for Obstructive Apnea: Strongly reduced flow volume for at least 7 seconds with MPS increase; adjustment range: 0-1 in steps of 0.1.

Weighting Factor for Obstructive Hypopnea: Reduced flow volume with inspiratory MPS increase; adjustment range: 0-1 in steps of 0.1.

Weighting Factor for Obstructive Event of First Degree: Significant cumulative MPS increase (beyond the aforementioned events); adjustment range: 0-1 in steps of 0.1.

Weighting Factor for Obstructive Event of Second Degree: Slightly pronounced cumulative MPS increase (beyond the aforementioned events); adjustment range: 0-1 in steps of 0.1.

Weighting Factor for Snoring: Cumulative inspiratory snoring with/without MPS increase or flattening; adjustment range: 0-1 in steps of 0.1.

Weighting Factor for Flattening: Cumulative flattening with/without MPS increase; adjustment range: 0-1 in steps of 0.1.

Weighting Factor Central for Apnea/Hypopnea: Adjustment range: −1 to 1 in steps of 0.1.

The pressure adjustment after events is carried out according to the selected ruleset. The event index is calculated after the expiration of the delay time for pressure increase or pressure decrease from the events that occurred during this period of time. The pressure is raised if the event index rises above one of two defined thresholds for pressure increase and the delay time before a renewed pressure increase has expired. The pressure is lowered if the event index falls below the defined threshold for the pressure reduction and the delay time for a renewed pressure reduction has expired. After a pressure increase or decrease, the event index and the counters for the period of time for calculating the index and for the delay times before a renewed pressure increase/reduction are set to 0.

The adjustment ranges pressure increase can vary, but in a preferred form, they are selected as follows:
event index 1: 0 . . . 30 [/h], resolution: 0.1 [/h]
event index 2: 0 . . . 30 [/h], resolution: 0.1 [/h]
pressure increase 1: 0 . . . 10 [hpa], resolution: 0.1 [hPa]
pressure increase 2: 0 . . . 10 [hPa], resolution: 0.1 [hPa]
delay time (=period of time for index calculation): 1 . . . 120 [minutes], resolution: 1 [minute].

To be able to conform quickly to the initially strong pressure increase requirement, two criteria for the pressure increase are defined, but the delay time of the two criteria is to be kept identical. FIG. 2 shows a corresponding screen display.

The adjustment ranges pressure reduction can vary, but in a preferred form, they are selected as follows:
event index: 0 . . . 30 [/h], resolution: 0.1 [/h]
pressure increase: 0 . . . 10 [hpa], resolution: 0.1 [hPa]
delay time (=period of time for index calculation): 1 . . . 120 [minutes], resolution: 1 [minute], split-night option, i.e., the titration can be interrupted after a defined time. If the split-night option is activated, the titration is terminated after expiration of the specified time. The recommended titration pressure is calculated and set. The device is then operated with this pressure until the user shuts the device off.

Adjustment Ranges: no interruption of the titration; 120 . . . 600 [minutes]; resolution: 5 [minutes]; (default: no split night).

During intervals affected by artifacts (e.g., with the mask open), the event detection is discontinued, and the index is frozen. The index calculation is reinitialized after an interval affected with artifacts of ≧5 minutes (adjustable).

Titration Pressure Calculation

The recommended titration pressure (in the mode titration CPAP) can be determined by the following methods if a titration time of at least 2 hours (adjustable) was maintained (including automatic control starting time); in this regard, time-related, event-related, and pressure-related values for a CPAP titration pressure are derived from the applied pressures according to the rules established above.

Pressure Percentile

The pressure variation of the specified period of time of the titration is analyzed. The pressure values are divided into 0.5-hPa classes, and the percent fraction (frequency) of the occurrence (of the pressure class) in the analysis time interval is determined. The classes are then accumulated, and the titration pressure is determined according to the specified percentile (smallest pressure value at which the percent fraction first exceeds the specified percentile). The pressure variations are shown in FIG. 3.

In a preferred form, the pressure values can be divided into the following (36) classes:
class 0: p<3 hPa
class 1: 3≦p<3.5 hPa
class 2: 3.5≦p<4 hPa
. . .
class 34: 19.5≦p<20 hPa
class 35: p≦20 hPa

The analysis time interval is given in % of the titration time (last n % of the titration time); value range: 10 . . . 100%; resolution: 5%, (default: 90%).

The percentile is given in percent; value range: 50 . . . 100%; resolution: 5%, (default: 95%).

Event Percentile

The pressure variation of the specified analysis time interval is analyzed. In a preferred embodiment, the pressure values during the occurrence of obstructive events (obstructive apnea, obstructive hypopnea, snoring, flattening) are divided into 0.5-mbar classes, the percent fractions are determined, and the titration pressure is determined from the cumulative values (smallest pressure value at which the percent fraction first exceeds the specified percentile). The adjustments are identical to the pressure percentile.

Final Pressure

In a preferred form, in the adjustment of the final pressure, the current (set) pressure when the “split-night time” is reached is used as the titration pressure and is stored as the recommended titration pressure.

Median

In a preferred embodiment, the pressure variation of the specified analysis time interval is analyzed, and the “mean value” (median) is fed back as the titration pressure.

The analysis time interval is specified in % of the titration time (last n % of the titration time); value range: 10 . . . 100%; resolution: 5%, (default: 90%).

Error Detection for Titration Pressure Calculation:

The function for calculating the titration pressure feeds back the determined titration pressure in the range of 40 . . . 200 (4 . . . 20 mbars). Values outside of this range are reserved as error codes.

In another especially preferred form of the invention, after the titration phase, another selection function can effect the downstream mode selection.

In one embodiment, the selection function can orient itself on the frequency of the events that have occurred per hour (e.g., the AHI) and/or the variability of the pressure values that develop from the diagnostic phase when events are triggered, and can select a setting of the initial parameters.

In addition, when an option is selected, the therapeutic recommendation can be realized directly in the need-dependent ventilation of the patient without preliminary checking by the user (automatic mode).

Pressure Adjustment in the Profile Mode

The pressure adjustment is carried out according to a selected pressure profile. Events do not lead to a pressure adjustment.

The pressure profile defines the pressure variation in the profile mode. In a preferred embodiment, 3 pressure profiles can be stored; a pressure profile can consist of a maximum of 50 segments, and each segment can contain the time since the device was started up and the pressure value. These can be varied in the respective bandwidths:
Profile ID: 1 . . . 3 (1, 2: fixed profiles, 3: user-defined profile)
Timer current position in the pressure profile
Number of segments used: 1 . . . 50
Segment duration (segment 1 . . . 50): 0 . . . 255 minutes, resolution: 1 minute
Initial pressure (segment 1 . . . 50): 2 . . . 18 hPa, resolution: 0.1 hPa
Final pressure (segment 1 . . . 50) 2 . . . 18 hPa, resolution: 0.1 hPa

As a specific example, the therapy recommendation already mentioned above can include the type within each form of therapy, the parameters, and a recommendation for man-machine interface and aerosol therapy.

In this regard, in an especially preferred form, the CPAP pressure and the slope of the softstart can be recommended by the device for the CPAP therapy.

In this regard, in an especially preferred form, the base pressure (average pressure) of the last night of therapy and the range of pressure variation (upper/lower pressure limit) can be recommended by the device for the APAP therapy.

In this regard, in an especially preferred form, the inspiratory and expiratory pressure level and thus the pressure difference, the inspiration time, and the expiration time can be recommended by the device for the bilevel therapy. In this regard, a safety interrogation can be made for minimal AMV, and the parameters can be adjusted to ensure sufficient ventilation for the patient.

At the end of the analysis phase, the device provides a statement about the suitability of the mask to be used for the patient, which can be determined from the events (e.g., leakages) that have occurred at different pressures. Selection can be made from among at least the following types: nasal pillow, nasal mask, full-face mask, full-head mask, and tube.

In another specific embodiment, it is proposed that the device be allowed to make an assignment/recommendation for a form of ventilation (CPAP, APAP, bilevel, other forms of ventilation) on the basis of at least two different parameters selected from the following group: flow (thermistor, dynamic pressure, flow restrictor, from therapeutic device), flow contour, effort (e.g., from sensors that detect excursions of the chest and abdomen), airway resistance (MPS or esophageal probe), plethysmogram, oxygen saturation, CO₂, EKG, EEG, EMG (chin and up to 2 leg EMG's), EOG, actimeter, microphone, video, body position, and light.

In one embodiment, it is proposed that the measurement of modulated pressure and flow values be carried out by a wide variety of sensors, such as piezoelectric, thermal, optical, acoustic, resistive, capacitive, and inductive.

Since the pressure during the expiratory phase (EPAP) keeps the airways open, in an especially preferred embodiment, the resistance is measured by MPS and a determination is made as to whether the lung is open.

In an especially preferred form of the invention, MPS is used to detect respiratory activity and respiratory phase, e.g., by expansion of the upper airways, etc.

In an especially preferred form of the invention, the bilevel difference, i.e., upper and lower pressure, is determined from MPS and the compliance. (bilevel titration mode)

In an especially preferred form of the invention, it is proposed that the MPS be used for the stimulation of receptors in order to provoke the triggering of a reflex arc by the sensitivity of the pharynx. The reflex arc results in reflex muscular splinting of the airways.

In another embodiment of the invention, it is contemplated that the respiratory work be derived from the MPS and that an evaluation of the respiratory effort be made by determination of the impedance. Determination of the respiratory drive or respiratory effort allows it to be used as the master parameter for controlling the therapeutic device.

In a preferred embodiment of the invention, the device supplies a message to the physician when the therapy is inadequate, e.g., via bluetooth, and stores this message in the device.

The device can be preset according to the diagnosis made by the physician and additionally allows continuous administration/supply of medications via the humidifier or nebulizer, so-called aerosol therapy, the purpose of which is the direct deposition of medications in the target organ, e.g., the deep airways. A preferred form of the invention is limited to bronchial diseases. The advantage of the inhalation of a drug instead of its administration as a tablet or by injection is that the substance quickly reaches the site of action and causes fewer undesired side effects in other organs.

The following means of producing aerosols are presently available: Compressed-air and ultrasonic nebulizers, propellant-driven metering aerosols, and dry-powder inhalers. The humidifier can be used in all device modes. It is activated or deactivated by pressing the humidifier key or via the serial interface. The humidifier level can be adjusted by the user by keyboard or serial interface. The humidifier is automatically deactivated as soon as the water reserve is exhausted or the humidifier is separated from the device. In this regard, the humidifier can be operated in at least six different humidifier stages and can display a warning notice when it is empty.

External appearance and embodiments of the connections, technical parameters:

The external design of the invention can be extremely flexible due to its modular composition of the sensors from sensor groups, which are connected by plug connectors, cables, or wirelessly with one another and with a memory unit and a visualization unit, which can be a PC or a PDA; in this regard, the configurability of the measurement electronics for different sensors (e.g., other amplification factors, filters, . . . ) is maintained.

As a special advantage of the invention, the configuration of the diagnostic system can be carried out by the PC or PDA via cable (directly or via a network) or wirelessly; operation is possible on the device itself with an operator interface.

In a preferred embodiment of the invention, it is possible to store the data in the device for subsequent evaluation, e.g., in the PC.

In another embodiment, the therapeutic device is provided with a memory, which can be easily removed (e.g., compact flash card) or can be read out remotely (by mobile radio, internet, telephone line, . . . ).

Power is supplied by battery or secondary cell. A self-test is performed, at least after the device has been turned on, and the state of battery charge is repeatedly monitored.

Furthermore, free channels are available for the analogous supply of other signals with or without amplification.

In a special embodiment, the quality of individual signals, e.g., as determined by impedance measurement of electrodes, is additionally displayed on the device by means of LED's or a display.

In another embodiment, it is proposed that a one-way EEG electrode and an associated amplifier circuit be combined with an A-D converter and an algorithm, which recognizes sleep stages (possibly by means of neural networks and fuzzy logic, cybernetics), to make it possible to react to the individual sleep stages with targeted parameter adjustments.

In addition, the therapeutic device can be provided with memories for patient data (e.g., weight, sex, diagnosis).

In an especially preferred embodiment, the therapeutic device can be provided with an identification number, with which it can uniquely identify itself in a network or when connected with a PC, so stored information (about servicing, patient, etc.) can be assigned to it from a data bank.

In another especially preferred embodiment, the device can be equipped with a telemetry unit for ambulant use, so that the diagnosis can already be made before a service provider drives back to the patient and can thus initiate therapy at once. In addition, the device can independently establish communication with a service site, e.g., for servicing or in the event of failure or to change the configuration.

Archiving Several Rulesets

During the reading in of the therapy parameters from the therapeutic device on the software side, the user is asked to store the ruleset if it is not consistent with any of the rulesets stored on the software side, and the therapeutic device is in the titration mode. Changes in the ruleset can be stored on the software side. The rulesets are stored, e.g., in an XML file in the directory, so that the stored rulesets can be fed to the device, called up, and printed out as a parameter list.

Both analog and digital signal output are possible (e.g., for polysomnography).

The control system of the device has access to the individual device units (fan, oscillation generator, humidifier, . . . ) and can modify their parameters as necessary.

The data storage includes the storage of the device data, calibration data, and titration/therapy data, as well as all other data necessary for operation and for deriving diagnostic values.

In another especially preferred embodiment, communication with external devices is possible via a serial interface and is also possible for operating the device with remote adjustment/PC and data export to the PC.

As a specific embodiment, the hardware environment can consist of various functional modules, which are illustrated in FIG. 4.

A multiple-line display is provided for the display. It is operated via the I2C interface of the microcontroller and contains, in addition, symbols for, e.g., filter change, service, humidifier, humidifier alarm, ventilation parameters and ventilation mode, softstart, pressure increase, pressure reduction and time, and the names and units of the displayed

Error messages are output via the device display.

In another preferred embodiment, the MPS raw signal can be sampled at 500 Hz, and then smoothing can be performed by (sliding) mean value formation over at least 3 values.

In another preferred embodiment, the pressure signal can be sampled at 100 Hz, and then smoothing can be performed by mean value formation over at least 100 values (new value after 1 s).

In another preferred embodiment, the humidifier voltage can be sampled at 50 Hz, and then smoothing can be performed by mean value formation over at least 5 values (new value after 0.1 s).

In another preferred embodiment, the (device) temperature can be sampled at 10 Hz, and then smoothing can be performed by mean value formation over 10 values (new value after 1 s).

In another preferred embodiment, the fan speed can be determined by a signal generated in the power board. The elapsed time of the pulses is measured with a timer unit (capture function) of the microcontroller and converted to a speed.

In another preferred embodiment, the speed of rotation of the oscillation generator (pump) can be determined by evaluation of the microphone signal. The microphone pulses (2 per rotation) are determined with a timer unit of the microcontroller over a period of 500 ms, and the current speed of rotation of the oscillation generator (pump) is computed from this.

In another preferred embodiment, a 4-channel D-A converter with a resolution of 10 bits can be used for the analog signal output. Two channels of the D-A converter are used for internal purposes (fan speed set value). The other two channels are used to output signals to external devices (e.g., polysomnograph). The signal to be output can be determined via the serial interface with the device-supporting software (modification of the corresponding EEPROM cell). The settings are stored in the EEPROM. Device-related and patient-related signals can be selected for output in the device, e.g., MPS, pressure, flow, and loss flow.

In a preferred embodiment, in the standard setting, the MPS signal and the flow signal can be output.

In another preferred embodiment, the humidifier output is controlled by PDM (pulse-duration modulation). The PDM is generated in the microcontroller (with a capture/compare unit) and relayed to the power board (via a port pin).

In another preferred embodiment, the fan is released or blocked by a port pin.

In another preferred embodiment, the speed of the oscillation generator (pump) is controlled by PDM. The PDM is generated in the microcontroller (with a capture/compare unit) and output (via a port pin).

In another preferred embodiment, the external real-time clock is operated. The date and clock time can be adjusted only in standby (offline) (via the serial interface or keyboard).

In another preferred embodiment, in the APAP mode, the memory of the device can store at least the lower pressure limit, upper pressure limit, rate of pressure increase, softstart pressure, and softstart time, and in the CPAP mode, it can store at least the CPAP, softstart pressure, and softstart time.

The recording time for the data of the last night is ≧8 hours. In addition, the calibration data of the pressure sensor are stored.

In another preferred embodiment, the annual compliance is recorded over a period of 366 days. The time of use (device turned on, pressure>threshold) per day in increments of 0.1 h is stored. A day is defined as 24 hours, with a new day beginning at 12:00 noon.

In a preferred embodiment, the humidifier compliance is recorded over a period of 366 days. The time of use (operation with humidifier) per day in increments of 0.1 h is stored. A day is defined as 24 hours, with a new day beginning at 12:00 noon.

In a preferred embodiment, the titration data are recorded over a period of a maximum of 10 hours. A maximum of 10 recordings (therapy starts) are stored. If more than 10 recordings are carried out, or if the maximum recording time is exceeded, the oldest entries or data are overwritten. Recordings with a duration<5 minutes are overwritten during the next device start.

In another preferred embodiment, the weekly compliance is recorded over a period of 30 days. Up to 30 therapy starts are recorded per day. A day is defined as 24 hours, with a new day beginning at 12:00 noon. Only days on which a titration/therapy was carried out are stored. After 30 entries, the oldest entry is overwritten. The following data are recorded per entry:

System-spanning interfaces in an especially preferred embodiment
for communication with external devices (PC, remote adjustment, test computer)
bluetooth
infrared
memory board
data export

The data export to the PC. In this regard, the PC (with the software) serves as master and requests the data from the device.

The device transmits the requested data, and at the end of the transmission a checksum is transmitted.

The transmission time for titration compliance and weekly compliance (together) cannot exceed 1 minute.

Device parameters can be requested/modified by means of a test computer during the testing via the serial interface (RS485).

In another ruleset for the autotitration CPAP, when arousals occur, the pressure can be increased more rapidly. During therapy monitoring, the lower pressure limit is selected below the pressure previously set for the patient in order to test the necessity of the higher pressure level. This can also be accomplished via a suitable adjusted pressure profile.

In another embodiment, the automatic pressure control is also carried out as a function of the current sleep stage or the sleep stage through which the patient has just passed.

In particular, the titration does not begin until the patient has fallen asleep and/or a pressure reduction is brought about in the nighttime waking phases of the patient.

The following functions are to be named, individually or in combination, as an additional preferred embodiment:

1. The autotitration CPAP or bilevel begins automatically as soon as a certain number of respiratory disturbances (e.g., 30 episodes of apnea) and/or arousals caused by such disturbances and/or incidents of low oxygen saturation and/or other pathological changes due to sleep-related respiratory disturbances have occurred.

2. The positive pressure ventilation is initiated at a higher pressure if the patient has problems falling asleep (“feeling of the patient that he is not getting enough air”). Common conditions associated with this problem are nasal congestion, severe obesity, and previous chronic treatment with CPAP.

If a few attempts at a slow increase in the pressure to a level sufficient to maintain airway patency are not tolerated, treatment is automatically switched to autotitration bilevel.

3. If persistent low arterial oxygen saturation values (SaO₂<88%) occur despite sufficient air flow in CPAP, an attempt is first made to increase the CPAP by 1-2 cm H₂O. If high levels of CPAP are already being used, bilevel ventilation is tried. If these efforts are unsuccessful, additional oxygen is supplied to the system in steps of 1 LPM until SaO₂>90%.

4. If the patient has problems tolerating nasal ventilation, accompanied by nasal constriction, a humidifier is automatically added. If the problems persist, the physician receives a message to administer a vasoconstrictor (vessel-constricting agents). Otherwise, the device outputs a recommendation for titration with a full-face mask.

5. If patients do not tolerate a level of CPAP pressure or bilevel pressure that is sufficient to stabilize the airways, a notice is output to elevate the head of the bed by 30°, or this is effected via an interface, or a recommendation that the patient be positioned on his side is output.

6. If high mouth leakages occur, the humidifier is turned on, and a recommendation to use a chin strap is output. If this remains unsuccessful, a change is made to bilevel ventilation or APAP to lower the average pressure. If this is unsuccessful, a recommendation to apply a full-face mask is displayed.

7. Bilevel titration is adjusted as follows: IPAP=EPAP are increased until obstructive apnea is no longer present. IPAP is then increased until hypopnea/snoring/low oxygen saturation are eliminated. If events occur despite maximum tolerated IPAP, EPAP is increased in increments of 1 cm H₂O until the events no longer occur and sufficient ventilation is present nevertheless.

Oxygen Titration:

1. If the patient is using oxygen to maintain sufficient arterial oxygen saturation during the day, supplemental oxygen is introduced at the accustomed flow rate at the beginning of the diagnostic study, unless the user sets its differently.

2. If low arterial oxygen saturation persists in the absence of apnea, hypopnea, or severe snoring, an oxygen titration is initiated—but only after an applicable diagnostic period, in order to document the necessity of oxygen administration. If the severe snoring occurs together with low oxygen saturation, CPAP therapy is attempted before oxygen is introduced.

3. If oxygen is added to a ventilation treatment or an oxygen titration is carried out due to low oxygen saturation that is not related to apnea/hypopnea, oxygen is titrated upward in increments of 1 LPM until the arterial oxygen saturation is >92% (>90% if greater than 3 LPM is required). There is a maximum value (e.g., 5 LPM) that can be automatically set by the device without physician consultation; higher values can be set manually.

Central Apnea:

1. If an obstructive or mixed apnea turns into central apnea of the Cheyne-Stokes type during the CPAP titration, continued increasing titration of the CPAP can be attempted. If no pressure level eliminates the central apnea, the pressure level is increased to a level that gets rid of the obstructive component or 10-12 cm H₂O, whichever is higher.

2. Short episodes of central apnea in REM sleep phases, which cause neither low oxygen saturation nor arousal, are not treated with a further increase in the CPAP by the device.

3. If frequent episodes of central apnea (not of the Cheyne-Stokes type) and subsequent arousals during a CPAP titration are detected by the device, a higher level of CPAP is used after prior demonstration of snoring/flattening. If this is not successful, a slightly lower CPAP level is tried (possible arousals as well as increased pressure/leakages).

The following functions are to be named, individually or in combination, as an additional preferred embodiment:

According to FIG. 1, the titration is begun with a predetermined initial pressure Pᵢₙᵢ1. Pressure upper limits 4 and lower limits 3 are preset. A rate of pressure increase 5 is also preset. In addition, the time after which the automatic control starts and ends can be adjusted by the user 2.

FIG. 6 shows a display of various adjustments to be made in the profile mode. This input screen can be used to set the pressure sequence with individual pressure sequences in duration, beginning, and applied pressure. It is thus possible for the user to adjust the pressure variations individually for each patient and to store them as a profile.

According to FIG. 7, different pressures 8 are supplied to the patient during the time tᵢ 9, depending on the weighted event indices 6 over a total time interval t 7. The rate of pressure increase 5 and minimum values for the time tᵢ 9 can be adjusted. During tᵢ, the applied pressure remains constant; the current event index is set back after each required pressure adjustment and is continuously recomputed during tᵢ. If an event index limit 10 is reached, a pressure adjustment 11 is made.

FIG. 8 shows the basic design of a ventilator. A respiratory gas pump is installed inside an apparatus housing 21, which has an operating panel 22 and a display 23. A connecting hose 25 is attached by a coupling 24. An additional pressure-measuring hose 26, which can be connected with the ventilator housing 21 by a pressure input connection 27, can run along the connecting hose 25. To allow data transmission, the ventilator housing 21 has an interface 28.

An expiratory element 29 is installed in an expanded area of the connecting hose 25 that faces away from the apparatus housing 21. An expiratory valve can also be used.

The drawing also shows a ventilator mask 30, which is designed as a nasal mask. The mask can be fastened on the patient's head by a head fastening device 31. A coupling device is provided in the expanded region of the ventilator mask 30 that faces the connecting hose 25.

1. A ventilator, which has a respiratory gas source that can be connected with a patient interface and a control unit designed for presetting at least two different ventilation modes, wherein the control unit is designed for the automatic determination, selection, and application of the various ventilation modes in at least some respiratory disorders according to a current patient-specific requirement.

2. A ventilator in accordance with claim 1, wherein the control unit is connected to at least one sensor for detecting a parameter.

3. A ventilator in accordance with claim 1, wherein the sensor is designed for detecting a physiological parameter.

4. A ventilator in accordance with claim 1, wherein the sensor is designed for detecting a device-specific parameter.

5. A ventilator in accordance with claim 1, wherein the control unit has an analyzer for signal analysis.

6. A ventilator in accordance with claim 1, wherein a ruleset is used to carry out a pressure adjustment.

7. A ventilator in accordance with claim 6, wherein the ruleset is fixed.

8. A ventilator in accordance with claim 6, wherein the ruleset is user-defined.

9. A ventilator in accordance with claim 1, wherein the control unit comprises an adaptation unit for changing the ventilation mode.

10. A ventilator in accordance with claim 1, wherein the control unit includes an artifact detection system.

11. A ventilator in accordance with claim 1, wherein the analyzer is designed to suppress a mode change when an artifact is detected.

12. A ventilator in accordance with claim 1, wherein the analyzer is designed for detecting inspiratory phases and expiratory phases.

13. A ventilator in accordance with claim 1, wherein the analyzer is designed to analyze at least one parameter related to the flow.

14. A ventilator in accordance with claim 1, wherein the analyzer is designed to analyze at least one parameter related to the pressure.

15. A ventilator in accordance with claim 1, wherein the analyzer has bandpass filtering.

16. A ventilator in accordance with claim 1, wherein the analyzer has a comparator that considers an individual patient history.

17. A ventilator in accordance with claim 1, wherein the analyzer has a comparator that considers preset parameter values.

18. A ventilator in accordance with claim 1, wherein the analyzer has a comparator that considers ideal values.

19. A ventilator in accordance with claim 1, wherein the control unit is designed with a fixed time interval for mode adaptation.

20. A ventilator in accordance with claim 1, wherein the control unit is designed with an adaptive time interval for mode adaptation.

21. A method for controlling a ventilator, which has a respiratory gas source that can be connected with a patient interface and a control unit, and in which at least two different modes of ventilation can be selectively activated by the control unit, wherein the various modes of ventilation are automatically determined, selected, and applied in at least some respiratory disorders according to a current patient-specific requirement.

22. A method in accordance with claim 21, wherein at least one measured value determined by a sensor is supplied to the control unit.

23. A method in accordance with claim 21, wherein a physiological measured value is determined by the sensor.

24. A method in accordance with claim 21, wherein a device-specific measured value is determined by the sensor.

25. A method in accordance with claim 21, wherein a current state of ventilation is evaluated by an analyzer.

26. A method in accordance with claim 21, wherein a pressure adjustment is carried out according to a ruleset.

27. A method in accordance with claim 26, wherein the pressure adjustment is carried out according to a fixed ruleset.

28. A method in accordance with claim 26, wherein the pressure adjustment is carried out according to a user-defined ruleset.

29. A method in accordance with claim 21, wherein the device control system is changed by an adaptation unit.

30. A method in accordance with claim 21, wherein the analyzer performs artifact detection.

31. A method in accordance with claim 21, wherein a change in the ventilation mode is suppressed by the control unit when an artifact is detected.

32. A method in accordance with claim 21, wherein the analyzer detects inspiratory phases and expiratory phases.

33. A method in accordance with claim 21, wherein the analyzer performs an analysis of at least one parameter related to flow.

34. A method in accordance with claim 21, wherein the analyzer performs an analysis of at least one parameter related to pressure.

35. A method in accordance with claim 21, wherein the analyzer performs a bandpass filtering.

36. A method in accordance with claim 21, wherein the analyzer evaluates an individual patient history.

37. A method in accordance with claim 21, wherein the analyzer evaluates preset parameter values.

38. A method in accordance with claim 21, wherein the analyzer evaluates ideal values.

39. A method in accordance with claim 21, wherein a required change in the mode of ventilation is checked at fixed time intervals.

40. A method in accordance with claim 21, wherein a required change in the mode of ventilation is checked at adaptive time intervals.

41. A method in accordance with claim 21, wherein, using a predetermined pressure profile, the current pressure is marked by a marker that accompanies it in time.

42. A method in accordance with claim 21, wherein adjustable weightings are assigned to the detected events, depending on their severity.

43. A method in accordance with claim 21, wherein, when an event occurs, it is supplied to a continuously updated index.

44. A method in accordance with claim 21, wherein, after an adjustable time and, at the same time, the pressure has risen above or fallen below a threshold value that can be set, a pressure adjustment is made.

45. A method in accordance with claim 21, wherein, after a titration, a selection function activates a mode.

46. A method in accordance with claim 21, wherein the measuring signals are continuously recorded.

47. A method in accordance with claim 21, wherein a diagnostic phase and a therapeutic phase are applied in the course of one night.

48. A method in accordance with claim 21, wherein the therapeutic pressure is determined and also applied in the course of one night.

49. A method in accordance with claim 21, wherein the titration pressure to be applied is formed by an evaluation function.

50. A method in accordance with claim 21, wherein, to carry out a pressure adjustment within a preset interval of time, a pressure is first held in an essentially constant interval and is then reduced essentially linearly, and that, as a reaction to the occurrence of an obstructive event, the pressure is raised by a preset difference, and then the same procedure is cyclically repeated.

51. A method in accordance with claim 21, wherein cumulative event values are evaluated during an evaluation phase of the titration.

52. A method in accordance with claim 21, wherein a type of mask to be connected with the ventilator is determined by the result of the titration evaluation.

53. A ventilator, which has a respiratory gas source that can be connected with a patient interface and a control unit designed for presetting at least two different ventilation modes, wherein a sensor for detecting at least one parameter is connected to the control unit, where the control unit is equipped with an event detection system, where the control unit has an analyzer for assigning a weighting to different classes of events, where the analyzer forms an event index within a predeterminable evaluation interval as a function of the frequency of the events per interval of time, and where the control unit predetermines a pressure change as a function of the exceeding of threshold values, such that different threshold values are assigned to different levels of ventilation pressure changes that are to be made.
